(19) United States
(12) Patent Application Publication
(10) Pub. No.: US 2011/0054174 A1
(43) Pub. Date: Mar. 3, 2011

(54) PROCESS FOR THE PREPARATION OF A GLUCOKINASE ACTIVATOR COMPOUND

(76) Inventors: Stephan Bachmann, Allschwil (CH); Alec Fettes, Zuerich (CH); Hans Iding, Rheinfelden (DE); Beat Wirz, Reinach BL (CH); Ulrich Zutter, Basel (CH)

(21) Appl. No.: 12/859,795

(22) Filed: Aug. 20, 2010

(30) Foreign Application Priority Data
Aug. 28, 2009 (EP) 09168909.1

(51) Int. Cl. C07D 241/20; C07C 233/01; C12P 17/12

(52) U.S. Cl. 544/407; 544/336; 564/224; 435/122

(57) ABSTRACT

The present invention relates to a process for the preparation of a compound of formula I,

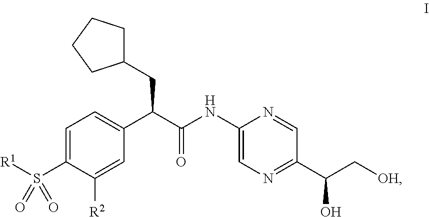

wherein R¹ is C₁₋₆-alkyl and R² is hydrogen or halogen.

(R)-2-phenyl propionic acid derivatives of formula I are key intermediates in the synthesis of 5-substituted-pyrazine or pyridine glucokinase activators of the formula Xa,

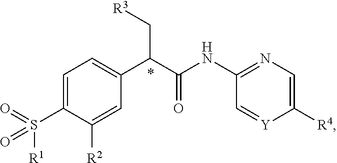

which have the potential to be useful for the treatment and/or prophylaxis of type II diabetes.

### PRIORITY TO RELATED APPLICATION(S)

This application claims the benefit of European Patent Application No. 09168909, filed Aug. 28, 2009, which is hereby incorporated by reference in its entirety.

### FIELD OF INVENTION

The object of the present invention was to develop an efficient synthetic pathway to compounds of formula I,

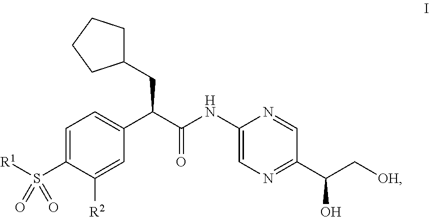

wherein R¹ is C₁₋₆-alkyl and R² is hydrogen or halogen.

(R)-2-phenyl propionic acid derivatives of formula I are key intermediates in the synthesis of 5-substituted pyrazine or pyridine glucokinase activators of formula Xa,

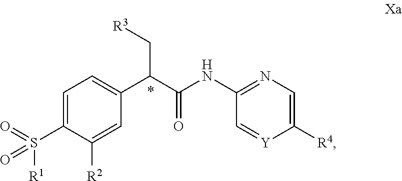

as disclosed in PCT International Patent Application No. WO 2004/052869 A1.
The glucokinase activators are useful for the treatment and/or prophylaxis of type II diabetes.

### SUMMARY OF THE INVENTION

The present invention relates in part to a process for the preparation of a compound of general formula I,

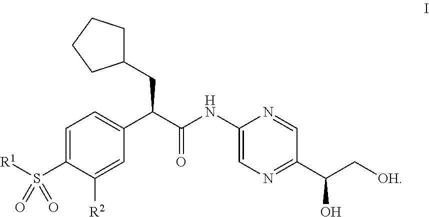

In addition, the invention relates to a compound of formula III,

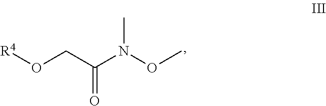

wherein R⁴ is t-butyl.

The invention further relates to a compound of formula IVa,

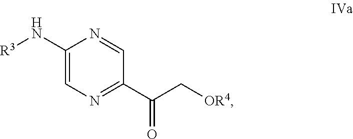

wherein R³ is an amino protecting group and R⁴ is a hydroxy protecting group.

Yet another aspect of the present invention is a compound of formula V,

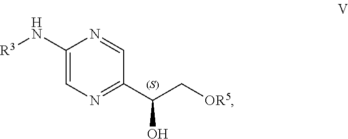

wherein R³ is an amino protecting group and R⁵ is H or is a hydroxy protecting group.

### DETAILED DESCRIPTION OF THE INVENTION

The object of the present invention was to develop an efficient synthetic pathway to prepare compounds of formula I

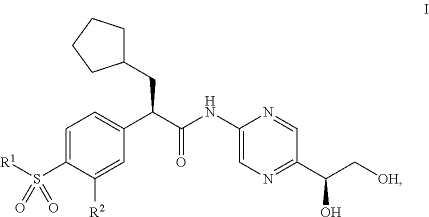

wherein R¹ is C₁₋₆-alkyl and R² is hydrogen or halogen.

The object could be achieved with the process of the present invention as outlined below, which process comprises the steps of

a) reacting a pyrazinamide of formula II,

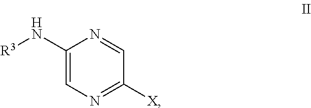

wherein R³ is an amino protecting group and X is halogen, with an amide compound of formula III,

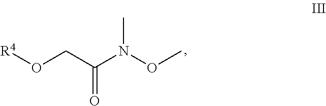

wherein R⁴ is a hydroxy protecting group, to form a ketone of formula IVa,

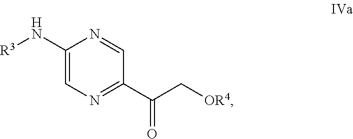

wherein R³ and R⁴ are as above;
b) optionally removing the R⁴ group to produce a ketone of formula IVb,

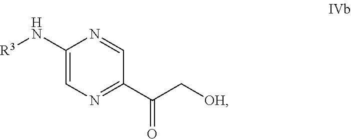

wherein R³ is as above, and asymmetrically reducing the compound of formula IVa or the compound of formula IVb to form the (S)-alcohol of formula V,

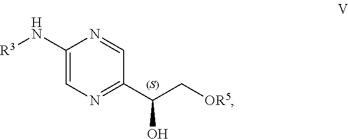

wherein R³ is as above and R⁵ is H or R⁴;
c) forming of the acetonide of formula VI,

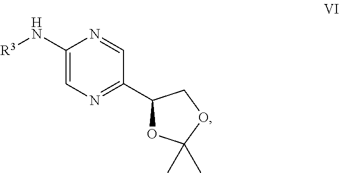

wherein R³ is as above;
d) removing R³ to form the amine of formula VII,

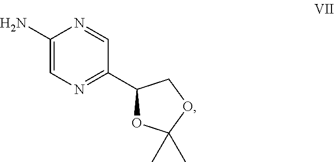

e) coupling said compound of formula VII with a (R)-2-phenyl propionic acid derivative of the formula VIII,

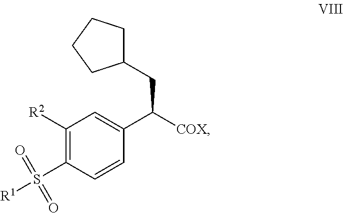

wherein R¹, R² and X are as above, to form the amide of formula IX,

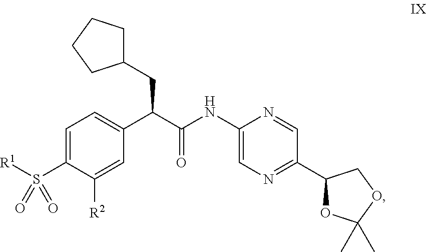

wherein R¹ and R² are as above; and
f) performing acidic acetonide hydrolysis to form the compound of formula I.

The following definitions are set forth to illustrate and define the meaning and scope of the various terms used to describe the invention herein.

The term “amino protecting group” refers to any substituents conventionally used to hinder the reactivity of the amino group. Suitable amino protecting groups are described in Green T., “Protective Groups in Organic Synthesis”, Chapter 7, John Wiley and Sons, Inc., 1991, 309-385. Preferred amino protecting groups as defined under R³ include pivaloyl, benyzyloxycarbonyl (Z), and fluorenylmethyloxycarbonyl (Fmoc), with pivaloyl being more preferred.

The term “hydroxy protecting group” refers to any substituents conventionally used to hinder the reactivity of the hydroxy group. Suitable hydroxy protecting groups are described in Green T., “Protective Groups in Organic Synthesis”, Chapter 2, John Wiley and Sons, Inc., 1991, 17-245. Preferred hydroxy protecting groups as defined under R⁴ include C₁₋₆-alkyl, C₁₋₆-alkylcarbonyl, C₁₋₆-alkoxy-C₁₋₆-alkyl and a saturated 5- or 6-membered heterocyclyl. More preferred are t-butyl, methoxymethyl, tetrahydropyranyl and benzyl. Even more preferred is t-butyl.

The term “C₁₋₆-alkyl”, alone or in combination with other groups, refers to a branched or straight-chain monovalent saturated aliphatic hydrocarbon radical of one to six carbon atoms, preferably one to four carbon atoms. This term is further exemplified by radicals as methyl, ethyl, n-propyl, isopropyl, n-butyl, s-butyl, t-butyl, pentyl, and hexyl and its isomers.

The term “halogen-C₁₋₆-alkyl” refers to a halogen substituted C₁₋₆-alkyl radical wherein halogen has the meaning as outlined below. Preferred “halogen-C₁₋₆-alkyl” radicals are the fluorinated

C₁₋₆-alkyl radicals such as CF₃, CH₂CF₃, CH(CF₃)₂, CH(CH₃)(CF₃), and C₄F₉.

The term “C₃₋₈-cycloalkyl” group refers to a cycloalkyl group containing from 3 to 8 carbon atoms, such as cyclopropyl, cyclobutyl, cyclopentyl, cyclohexyl, cycloheptyl and cyclooctyl.

The term “C₁₋₆-alkoxy” refers to a branched or straight-chain monovalent saturated aliphatic hydrocarbon radical of one to six carbon atoms, preferably 1 to 4 carbon atoms attached to an oxygen atom. Examples of “alkoxy” are methoxy, ethoxy, propoxy, isopropoxy, isobutoxy, t-butoxy and hexyloxy. Preferred are the alkoxy groups specifically exemplified herein.

The alkyl chain of the alkoxy group can optionally be substituted, particularly mono-, di- or tri-substituted by alkoxy groups as defined above, preferably methoxy or ethoxy, or by aryl groups, preferably phenyl. Preferred substituted alkoxy group is the benzyloxy group.

The term “C₁₋₆-alkyl carbonyl” refers to a C₁₋₆-alkyl substituted carbonyl group, preferably to a C₁₋₄-alkycarbonyl group. It includes for example acetyl, propanoyl, butanoyl and pivaloyl. A preferred alkyl carbonyl group is acetyl.

The term “C₁₋₆-alkyl carbonyl oxy” refers to a C₁₋₆-alkyl carbonyl substituted —O— group, preferably to a C₁₋₄-alkyl carbonyl substituted —O— group.

The term “mono- or di-C₁₋₆-alkyl-amino” refers to an amino group, which is mono- or disubstituted with C₁₋₆-alkyl, preferably C₁₋₄-alkyl. A mono-C₁₋₆-alkyl-amino group includes for example methylamino and ethylamino. The term “di-C₁₋₆-alkyl-amino” includes for example dimethylamino, diethylamino and ethylmethylamino. Preferred are the mono- and di-C₁₋₄-alkylamino groups specifically exemplified herein. It is hereby understood that the term “di-C₁₋₆-alkyl-amino” includes ring systems wherein the two alkyl groups together with the nitrogen atom to which they are attached form a 4 to 7 membered heterocycle which also may carry one further hetero atom selected from nitrogen, oxygen or sulfur.

The term “aryl”, alone or in combination with other groups, relates to a phenyl or naphthyl group, which can optionally be mono-, di-, tri- or multiply-substituted by halogen, hydroxy, CN, halogen-C₁₋₆-alkyl, NO₂, NH₂, NH(C₁₋₆-alkyl), N(C₁₋₆-alkyl)₂, carboxy, aminocarbonyl, C₁₋₆-alkyl, alkoxy, C₁₋₆-alkyl carbonyl, C₁₋₆-alkylsulfonyl, SO₂-aryl, SO₃H, SO₃-alkyl, SO₂—NR′R″, aryl and/or aryloxy. A preferred aryl group usually is phenyl, however the preference for aryl may differ as indicated hereinafter for certain substituents.

The term “saturated 5- or 6-membered heterocyclyl” refers to a saturated heterocyclic group with one or two heteroatoms selected from oxygen and nitrogen. Preferred saturated 5- or 6-membered heterocyclic groups are tetrahydropyranyl and tetrahydrofuranyl, preferably tetrahydropyranyl.

The term “halogen” refers to a fluorine, chlorine, bromine or iodine atom, preferably to a chlorine atom.

### Step a)

Step a) involves the reaction of a pyrazinamide of formula II with an amide compound of the formula III to form a ketone of formula IVa.

Pyrazinamides of formula II can be prepared by protecting the amino group of a commercially available 5-halogen-2-amino pyrazine following methods known to those skilled in the art.

In a preferred pyrazinamide of formula II, R³ is pivaloyl and X is halogen, preferably chlorine or bromine, more preferably bromine. The preparation of a preferred pyrazinamide of formula II is outlined in the scheme 1 below.

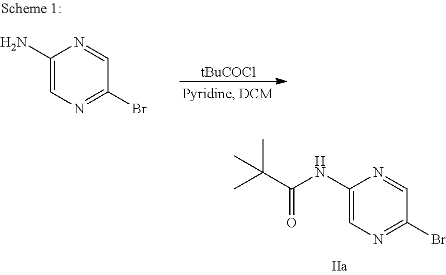

The amide compounds of formula III can be prepared e.g. as outlined in the scheme below.

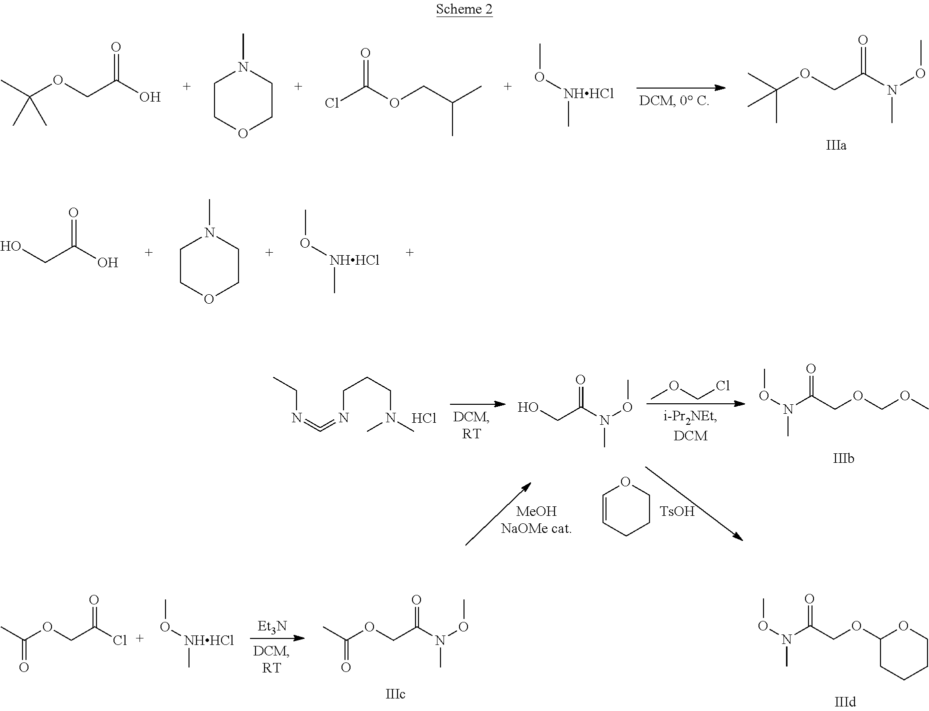

The amide compound of formula III,

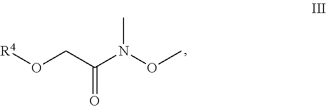

wherein R⁴ is t-butyl is so far not known in the art and therefore represents a further embodiment of the present invention.

The conversion of compound II to compound IVa can as a rule be performed in the presence of a lithiating agent, for example with 2.0 to 2.2 equivalents, preferably 2.1 equivalents of a lithiating agent which can be selected from common alkyl lithium compounds, preferably n-butyl lithium followed by the addition of the amide III.

The reaction is performed in an organic solvent, preferably in ethers like diethylether, dimethoxyethane, tetrahydrofuran and methyltetrahydrofuran, more preferably in tetrahydrofuran.

The reaction temperature can be selected in the range of from −60° C. to −100° C., preferably from −90° C. to −100° C.

The ketone compound of formula IVa,

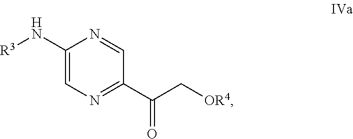

wherein R³ is an amino protecting group and R⁴ is a hydroxy protecting group, represents a further embodiment of the present invention.

### Step b)

Step b) involves the asymmetric reduction of the ketone of formula IVa to form the (S)-alcohol of the formula V. Optionally, the R⁴ hydroxy protecting group of the ketone of formula IVa may be first removed to produce a ketone of formula IVb which then undergoes the aforementioned asymmetric reduction.

The removal of the hydroxy protecting group in the ketone of formula IVa and the formation of the ketone of formula IVb is a procedure commonly known in the art.

The ketone IVa or IVb can be reduced by an enzymatic or microbial asymmetric reduction catalyzed by an oxidoreductase, for example one dependent on NADH or HADPH, or by a catalytic asymmetric hydrogenation using a metal complex catalyst.

### Enzymatic or Microbial Asymmetric Reduction by an Oxidoreductase:

The asymmetric reduction is catalyzed by an oxidoreductase, usually in the presence of NADH or NADPH as cofactor, which is regenerated in-situ.

The oxidized cofactor is as a rule continuously regenerated with a secondary alcohol as a cosubstrate. Typical cosubstrates can be selected from 2-propanol, 2-butanol, 2-pentanol, 4-methyl-2-pentanol, 2-heptanol and 2-octanol, preferably 2-propanol, 4-methyl-2-pentanol and 2-octanol. Preferably, the cofactor is regenerated by means of the cosubstrate at the same enzyme also catalyzing the target reaction.

Also well-known is the cofactor regeneration via an additional enzyme oxidizing its natural substrate and providing the reduced cofactor. For example secondary alcohol dehydrogenase/alcohol; glucose dehydrogenase/glucose; formate dehydrogenase/formic acid; glucose-6-phosphate dehydrogenase/glucose-6-phosphate; phosphite dehydrogenase/phosphite; hydrogenase/molecular hydrogen and the like. In addition electrochemical regeneration methods are known as well as chemical cofactor regeneration methods comprising a metal catalyst and a reducing agent.

Preferred microbial oxidoreductase enzymes originate from yeasts, bacteria or from mammalian cells.

A preferred representative of an oxidoreductase with bacterial origin is the NADH dependent oxidoreductase from IEPox28 (DSM 22053). This microorganism has been deposited by IEP GmbH, Wiesbaden, Germany according to the Budapest Treaty with the Deutsche Sammlung von Mikroorganismen und Zellkulturen GmbH (DSMZ), Braunschweig, Germany on Dec. 1, 2008.

A preferred representative of an oxidoreductase with yeast origin is the NADPH dependent oxidoreductase from a Candida strain IEPox63 (DSM 22052). This microorganism has been deposited by IEP GmbH, Wiesbaden, Germany according to the Budapest Treaty with the Deutsche Sammlung von Mikroorganismen und Zellkulturen GmbH (DSMZ), Braunschweig, Germany on Dec. 1, 2008.

Further preferred representatives of oxidoreductases with yeast origin are the oxidoreductases from Candida magnolia as disclosed in the PCT Int. Publication WO 2007/033928 and identified as SEQ ID No2 and SEQ ID No4.

A preferred representative of an oxidoreductase with mammalian cell origin is the NADPH dependent oxidoreductase from IEPox19 (DSM 22167). This microorganism has been deposited by IEP GmbH, Wiesbaden, Germany according to the Budapest Treaty with the Deutsche Sammlung von Mikroorganismen and Zellkulturen GmbH (DSMZ), Braunschweig, Germany on Jan. 14, 2009.

A preferred example for an isolated enzyme is the NADPH dependent oxidoreducatse KRED-NADPH-101 (commercially available from Codexis Inc, Redwood City, Calif., USA).

Optionally, the asymmmeric reduction can be performed in the presence of an organic cosolvent which can be selected from, for example, glycerol, diethylether, tert.butylmethylether, diisopropylether, dibutylether, ethylacetate, butylacetat, heptane, hexane or cyclohexene.

The reaction temperature is usually kept in a range between 1° C. and 50° C., preferably between 20° C. and 35° C.

Upon termination of the reaction (as a rule >90% conversion) the product is conventionally worked up by extraction and—if required—prior filtration of the biomass.

Depending on the ketone substrate the most preferred catalyst/cofactor/cosubstrate systems vary.

For the preferred ketone of formula IVa with R³=pivaloyl and R⁴=t-butyl the preferred systems are:

| Oxidoreductase | Cofactor | Cosubstrate |
|---|---|---|
| IEPox19 | NADPH | 2-octanol |
| IEPox28 | NADH | 2-propanol |
| WO 2007/033928 | NADPH | 2-methyl-4-pentanol |
| SEQID No 2 | | |

For the preferred ketone of formula IVa with R³=pivaloyl and R⁴=tetrahydropyranyl the preferred systems are:

| Oxidoreductase | Cofactor | Cosubstrate |
|---|---|---|
| IEPox19 | NADPH | 2-octanol |
| IEPox28 | NADH | 2-propanol |
| WO 2007/033928 | NADPH | 2-methyl-4-pentanol |
| SEQID No 4 | | |

For the preferred ketone of formula IVa with R³=pivaloyl and R⁴=methoxymethyl the preferred systems are:

| Oxidoreductase | Cofactor | Cosubstrate |
|---|---|---|
| IEPox19 | NADPH | 2-octanol |
| IEPox28 | NADH | 2-propanol |
| IEPox63 | NADPH | 2-methyl-4-pentanol |
| WO 2007/033928 | NADPH | 2-methyl-4-pentanol |
| SEQID No 2 | | |
| WO 2007/033928 | NADPH | 2-methyl-4-pentanol |
| SEQID No 4 | | |
| KRED-NADPH-111 | NADPH | glucose/GDH |

For the preferred ketone of formula IVb with R³=pivaloyl the preferred system is:

| Oxidoreductase | Cofactor | Cosubstrate |
|---|---|---|
| KRED-NADPH-101 | NADP | glucose/GDH |

Optionally, the isolated enzyme(s) or the whole cells might be immobilized by one of the numerous conventional methods described in literature and used in immobilized form.

Catalytic asymmetric hydrogenation using a metal complex catalyst: Alternatively the ketone of formula IV can also be subjected to a catalytic asymmetric hydrogenation using a metal complex catalyst.

As a rule Ruthenium-, Iridium- or Rhodium metal complex catalysts selected from compounds of formulas

Ru(Z)₂D (XXa);

[Ru(Z)₂₋ₚ(D)(L)ₘ](Y)ₚ (XXb);

[Ru(D)(L)₂](Y)₂ (XXc);

[M(D)LX] (XXd); and

[M(D)L]⁺Y⁻ (XXe);

wherein
Z is selected from the group consisting of: hydrogen, halogen, η⁵-2,4-pentadienyl, η⁵-2,4-dimethyl-pentadienyl and the group A-COO⁻, wherein
A is selected from the group consisting of C₁₋₆-alkyl, aryl, halogenated C₁₋₆-alkyl and halogenated aryl;
Y is a non-coordinating anion;
D is a chiral phosphine ligand;
L is a neutral ligand;

### M is Iridium or Rhodium

X is a halogen atom;
m is an integer from 1 to 3; and
p is 1 or 2;
can be applied.

In a preferred embodiment the phosphine ligand D is selected from

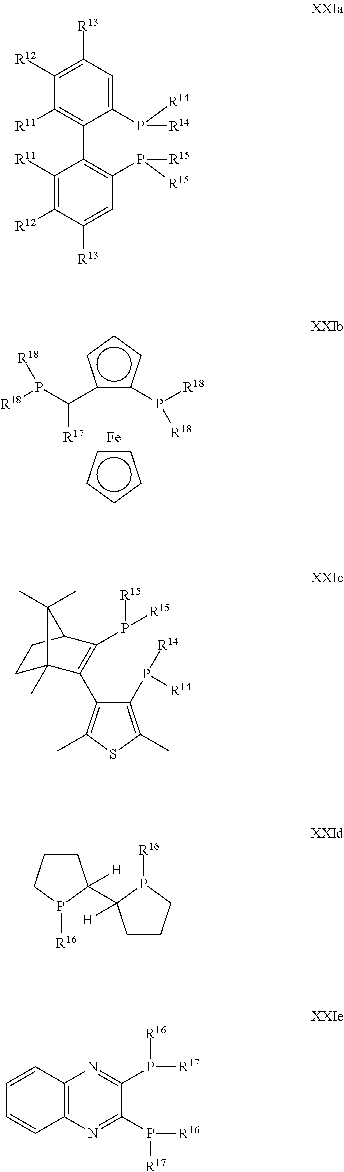

wherein
R¹¹ is selected from the group consisting of C₁₋₆-alkyl, C₁₋₆-alkoxy, hydroxy and C₁₋₆-alkyl carbonyl oxy;
R¹² and R¹³ independently of each other are selected from the group consisting of hydrogen, C₁₋₆-alkyl, C₁₋₆-alkoxy and di-(C₁₋₆-alkyl)amino; or
R¹¹ and R¹² which are attached to the same phenyl group or
R¹² and R¹³ which are attached to the same phenyl group, taken together, are —X—(CH₂)ᵣ—Y—,
wherein X is —O— or —C(O)O—, Y is —O— or —N(C₁₋₆-alkyl)- and r is an integer from 1 to 6, or a CF₂ group,
or both R¹¹s, taken together, are —O—(CH₂)ᵣ—O— or O—CH(CH₃)—(CH₂)ᵣ—CH(CH₃)—O—, wherein r is an integer from 1 to 6, or
R¹¹ and R¹², or R¹² and R¹³, together with the carbon atoms to which they are attached, form a naphthyl, tetrahydronaphthyl or dibenzofuran ring;
R¹⁴ and R¹⁵ independently of each other are selected from the group consisting of C₁₋₆-alkyl, C₃₋₈-cycloalkyl, phenyl, napthyl and heteroaryl, substituted with 0 to 7, preferably 0 to 5 substituents independently selected from the group consisting of C₁₋₆-alkyl, C₁₋₆-alkoxy, di(C₁₋₆-alkyl)amino, morpholino, phenyl and tri(C₁₋₆-alkyl)silyl, carboxy, and C₁₋₆-alkoxycarbonyl;
R¹⁶ is C₁₋₆-alkyl;
R¹⁷ is C₁₋₆-alkyl;
R¹⁸ is selected from the group consisting of aryl, heteroaryl, C₃₋₈-cycloalkyl and C₁₋₆-alkyl.
Y is preferably selected from the group consisting of halides, AsF₆⁻, BF₄⁻, ClO₄⁻, SbF₆⁻, PF₆⁻, B(phenyl)₄⁻, B(3,5-di-trifluoromethyl-phenyl)₄⁻, CF₃SO₃⁻, and C₆H₅SO₃⁻.
Y more preferably is selected from the group consisting of BF₄⁻, B(3,5-di-trifluoromethyl-phenyl)₄⁻ and CF₃SO₃⁻.
L is preferably selected from the group consisting of ethylene, propylene, cyclooctene, 1,3-hexadiene, 1,5-hexadiene, bicyclo-[2.2.1]hepta-2,5-diene, (Z,Z)-1,5-cyclooctadiene, benzene, hexamethylbenzene, 1,3,5-trimethylbenzene, p-cymene and solvents selected from tetrahydrofuran, N,N-dimethylformamide, acetonitrile, dimethylsulfoxide, benzonitrile, acetone, methanol and pyridine.

If L is a ligand comprising two double bonds, e.g. 1,5-cyclooctadiene, only one such L is present. If L is a ligand comprising only one double bond, e.g. ethylene, two such Ls are present. L more preferably is (Z,Z)-1,5-cyclooctadiene.

X is a halide such as Cl⁻, Br⁻ or I⁻, preferably Cl⁻.
A preferably is CH₃COO⁻ or CF₃COO⁻.

Preferred metal complex catalysts have the formula

[M(D)L]⁺Y⁻ (XXe)

wherein M, D, L, and Y are as described above.

Even more preferred are the catalysts of formula XXe having the following variable groups.

| Catalyst | | | | | | |
|---|---|---|---|---|---|---|
| Type | M | Z | D | L | Y | X |
| XXe/Rh-1 | Rh | — | XXIa | COD | BF₄ | — |
| XXe/Rh-2 | Rh | — | XXIb | COD | BF₄ | — |
| XXe/Rh-3 | Rh | — | XXIe | COD | BF₄ | — |
| XXe/Rh-4 | Rh | — | XXIa | COD | BARF | — |
| XXe/Rh-5 | Rh | — | XXIa | COD | OTf | — |
| XXe/Rh-6 | Rh | — | XXIc | COD | OTf | — |
| XXe/Rh-7 | Rh | — | XXId | COD | BF₄ | — |
| XXe/Ir-1 | Ir | — | XXIa | COD | BARF | — |

For the preferred ketone of formula IVa with R³=pivaloyl and R⁴=tert. butyl the preferred metal complex catalyst is:

[Rh(COD)((S)-3,5-tBu,4-MeO-MeOBIPHEP)]BF₄.

For the preferred ketone of formula IVa with R³=pivaloyl and R⁴=methoxymethyl the preferred metal complex catalyst is:

[Rh(COD)((S)-3,5-tBu,4-MeO-MeOBIPHEP)]BF₄.

In the ruthenium metal complex catalysts of the formula XXa, XXb and XXc the ruthenium is characterized by the oxidation number II. These complexes can in principle be prepared in a manner known per se. They can be isolated or used directly (in situ preparation) e.g. according to B. Heiser et al., Tetrahedron: Asymmetry 1991, 2, 51 or N. Feiken et al., Organometallics 1997, 16, 537 or J.-P. Genet, Acc. Chem. Res. 2003, 36, 908 or K. Mashima et al., J. Org. Chem. 1994, 53, 3064, M. P. Fleming et al., U.S. Pat. No. 6,545,165 B1, and references cited therein as well as O. Briel et al. in Catalysis of Organic Reactions, CRC Press, Boca Raton, 2009 specifically for the ferrocene-based Ru-complexes.

In the rhodium and iridium metal complex catalysts of the formula XXd and XXe the metal is characterized by the oxidation number I. They can be prepared, for example, by reaction of metal precursors such as e.g. di-η⁴-chloro-bis[η⁴-4-(Z,Z)-1,5-cyclo-octadiene]dirhodium(I) ([Rh(cod)Cl]₂), di-μ-chloro-bis[η⁴-norbornadiene]-dirhodium(I) ([Rh(nbd)Cl]₂), bis[η⁴-(Z,Z)-1,5-cyclooctadiene]rhodium tetra-fluoroborate ([Rh(cod)₂]BF₄) or bis[η⁴-(Z,Z)-cyclooctadiene]rhodium perchlorate ([Rh(cod)₂]ClO₄) or the corresponding Iridium analogues with a chiral phosphine ligand in a suitable inert organic or aqueous solvent (e.g. according to the methods described in J. Am. Chem. Soc. 1971, 93, 2397 or E. Jacobsen, A. Pfaltz, H. Yamamoto (Eds.), Comprehensive Asymmetric Catalysis I-III, Springer Verlag Berlin (1999) and references cited therein).

Rhodium or iridium metal complex catalysts as described above can also be prepared in situ, i.e. just before use and without isolation. The solution in which such a catalyst is prepared can already contain the substrate for the enantioselective hydrogenation or the solution can be mixed with the substrate just before the hydrogenation reaction is initiated.

In general the asymmetric hydrogenation is performed in an organic solvent at a reaction temperature between 10° C. and 100° C., preferably 20° C. to 80° C. and a pressure between 1 and 150 bar, preferably between 10 bar and 80 bar.

The substrate/catalyst ratio (S/C) is commonly between 5 and 100,000, preferentially between 100-50,000.

For Rh and Ir-type catalysts the S/C ratio as a rule ranges from 20 to 2000.

Suitable solvents for the hydrogenation with ruthenium metal complexes are alcohols, hydrocarbons, chlorinated hydrocarbons, fluorinated and polyfluorinated aliphatic or aromatic hydrocarbons, supercritical or liquid carbon dioxide, THF, water and mixtures thereof. Preferred solvents are alcohols, preferably methanol, and chlorinated hydrocarbons, preferably methylene chloride and THF.

Suitable solvents for iridium and rhodium metal complexes are alcohols; aromatic hydrocarbons, such as benzene, toluene, and trifluorotoluene; halogenated hydrocarbons, such as dichloromethane, dichlororethane, etc.; polyalcohols such as ethylene glycol; amides such as DMF, DMA, and N-methylpyrrolidinone; supercritical or liquid carbon dioxide; acetonitrile; water; and DMSO. Preferred solvents are alcohols, such as ethanol, trifluoro ethanol or chlorinated hydrocarbons such as methylene chloride.

The solvents can be used alone or as mixture of solvents mentioned above.

The (S)-alcohols of the formula

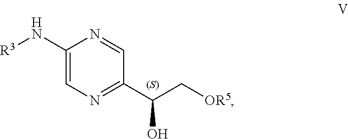

wherein R³ is an amino protecting group and R⁵ is H or has the meaning of R⁴ are compounds so far not known in the art and therefore represent a further embodiment of the present invention.

In a preferred (S)-alcohol of formula V, R³ is pivaloyl and R⁵ is selected from the group consisting of H, C₁₋₆-alkyl, C₁₋₆-alkylcarbonyl, C₁₋₆-alkoxy-C₁₋₆-alkyl and a saturated 5- or 6-membered heterocyclyl. R⁵ is preferably H or t-butyl.

### Step c)

Step c) involves the conversion of the (S)-alcohol of formula V to the acetonide of formula VI.

The reaction can be performed in accordance with the disclosure in the PCT Int. Publication WO 2004/052869, page 162, line 19 to page 163, line 8.

The conversion is preferably performed by acidic treatment of the compound of formula V with trifluoroacetic acid and subsequent treatment with 2,2-dimethoxypropane.

In a preferred ketal of formula VI, R³ is pivaloyl.

### Step d)

Step d) involves the removal of the amino protecting group R³ in compound VI to form the amine of formula VII.

The reaction can be performed in accordance with the disclosure in the PCT Int. Publication WO 2004/052869, page 163, line 10 to line 22, as a rule in the presence of a base.

Suitable bases are alcoholic solutions of an alkali carbonate or of an alkali hydroxide preferably of an aqueous sodium hydroxide.

As a rule the aqueous sodium hydroxide is used in combination with an organic solvent, preferably with tetrahydrofuran at elevated temperature, preferably at about 70° C.

The amine of formula VII can be isolated by extraction, e.g. with dichloromethane.

### Step e)

Step e) involves the coupling of the amine of formula VII with a (R)-2-phenyl propionic acid derivative of the formula VIII to form the amide of formula IX to form the amide of formula IX.

The reaction can be performed in accordance with the disclosure in the PCT Int. Publication WO 2004/052869, page 163, line 24 to page 164, line 11, as a rule in the presence of a suitable organic solvent such as methylene chloride and a tertiary amine such as pyridine.

The amide of formula IX can be isolated usually by an aqueous work up procedure, preferably using dichloromethane as organic solvent.

In the preferred amide of formula IX R¹ is methyl and R² is chlorine.

### Step f)

Step f) involves acidic acetonide hydrolysis to form the compound of formula I.

The reaction can be performed in accordance with the disclosure in the PCT Int. Publication WO 2004/052869, page 164, line 13 to line 26.

A suitable acid for the ketal ring opening is an aqueous mineral acid, preferably an aqueous hydrochloric acid.

The compound of formula I can be isolated usually by an aqueous work up procedure, preferably using dichloromethane as organic solvent.

Further purification of the compound of formula I can happen by crystallization in a suitable organic solvent or mixtures thereof, preferably in a mixture of isopropyl acetate and hexane or heptane.

In the preferred compound of formula I, R¹ is methyl and R² is chlorine.

### EXAMPLES

### Step a

### Example 1

### a) tert-Butoxyacetic acid

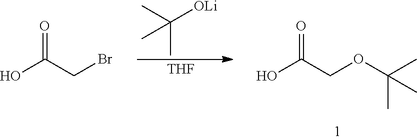

To a solution of 20% Lithium tert-butoxide in THF (421.0 g, 1.05 mol) was added while stirring a solution of bromoacetic acid (69.5 g, 0.50 mol) in THF (50 ml) over 30 min. After refluxing for 1 h, ˜250 ml THF was distilled off and the brownish reaction mixture was cooled to RT. The reaction mixture was diluted with 250 ml TBME and acidified under cooling with 200 ml 3 M HCl (note 7). The organic layer was washed twice with 125 ml 10% brine and all aqueous layers were extracted with 125 ml TBME. The combined organic layers were dried (Na₂SO₄) and evaporated to dryness affording 64.9 g (98.2%) crude tert-butoxyacetic acid 1 as a brown oil which was used without purification in the next step. ¹H NMR (CDCl₃, 400 MHz) δ 1.27 (s, 9H), 4.03 (s, 2H), 8.50 (br s, 1H).

### b1) 2-tert-Butoxy-N-methoxy-N-methyl-acetamide

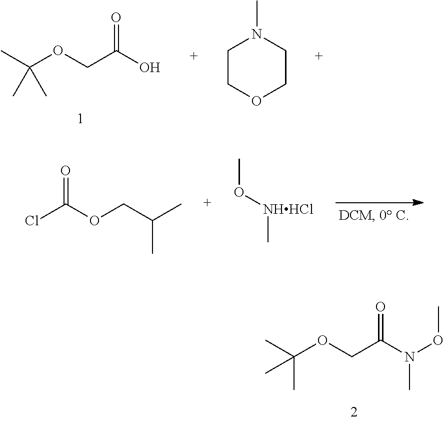

A pre-formed solution of tert-butoxyacetic acid 1 (26.4 g, 200 mmol) and 4-methylmorpholine (20.2 g 200 mmol) in 50 ml dichloromethane was added at 0° C. to a solution of isobutyl chloroformiate (27.3 g 200 mmol) in 150 ml dichloromethane. After additional stirring at 0° C. for 30 min, N,O-dimethylhydroxylamine hydrochloride (21.5 g, 220 mmol) was added all at once followed by the drop wise addition of 4-methylmorpholine (24.3 g, 240 mmol) over ˜30 min. Stirring at 0° C. was continued for 30 min and at RT for 30 min. The reaction mixture was washed with 100 ml 1 M HCl and 100 ml 10% brine. Both aqueous layers were extracted with 100 ml dichloromethane and the combined organic layers were dried (Na₂SO₄) and evaporated to dryness affording 34.7 g (“99.1%”) brown oil. Vacuum distillation over a 18 cm Vigreux column yielded 29.9 g (85.3%) Weinrebamide 2 as a colorless oil, bp. 71° C./0.5 mbar. ¹H NMR (CDCl₃, 400 MHz) δ 1.26 (s, 9H), 3.19 (s, 3H), 3.71 (s, 3H), 4.17 (s, 2H).

### b2) 2-Hydroxy-N-methoxy-N-methyl-acetamide

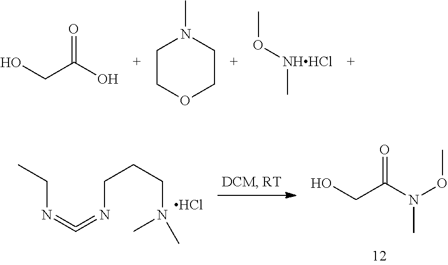

To a solution of 4-methylmorpholine (44.5 g, 440 mmol) in 500 ml dichloromethane was added while stirring glycolic acid (33.5 g, 440 mmol) and N,O-dimethylhydroxylamine hydrochloride (39.0 g, 400 mmol). The white suspension was cooled to 0° C. and 1-(3-dimethylaminopropyl)-3-ethylcarbodiimide hydrochloride (84.4 g, 440 mmol) were added all at once. After stirring at 0° C. for 1 h and at RT for 24 h the suspension was washed with 2M HCl (50 ml) and deionized water (50 ml). Both aqueous layers were extracted with dichloromethane (500 ml) and the combined organic layers were dried (Na₂SO₄) and evaporated to dryness affording 43.2 g (90.6%) crude product as a colorless liquid. Vacuum distillation over a 10 cm Vigreux column afforded 38.4 g (80.6%) Weinreb-amide 12 as a colorless oil, bp. 59-60° C./0.5 mbar. ¹H NMR (CDCl₃, 400 MHz) δ 3.12 (s, 1H), 3.25 (s, 3H), 3.70 (s, 3H), 4.29 (s, 2H).

### b3) 2-Acetoxy-N-methoxy-N-methyl-acetamide

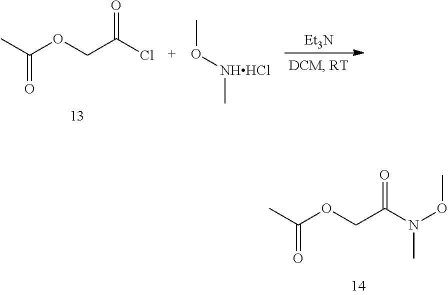

To a solution of acetoxyacetyl chloride 13 (68.3 g, 500 mmol) in 500 ml dichloromethane were added while stirring N,O-dimethylhydroxylamine hydrochloride (51.2 g, 525 mmol) all at once. The white suspension was cooled to 0° C. and triethylamine (106.2 g, 1050 mmol) was added at 0° C. over 1 h. After additional stirring at RT for 1 h, the white suspension was washed with 0.5M HCl (500 ml) and deionized water (500 ml). Both aqueous layers were extracted with 250 ml dichloromethane and the combined organic layers were dried and evaporated to dryness affording 76.3 g (94.7%) crude 14 as a pale yellow liquid which was used without purification in the next step. ¹H NMR (CDCl₃, 400 MHz) δ 2.18 (s, 3H), 3.20 (s, 3H), 3.74 (s, 3H), 4.82 (s, 2H).

### b4) 2-Hydroxy-N-methoxy-N-methyl-acetamide

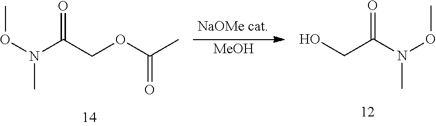

To a solution of 2-acetoxy-N-methoxy-N-methyl-acetamide 14 (80.6 g 500 mmol) in methanol (1000 ml) were added while stirring 1.0M sodium methoxide in methanol (5 ml, 5 mmol) and the pale yellow solution was stirred at RT for 3 h. After the addition of 37% HCl (0.45 ml, ca. 5 mmol) the reaction mixture was evaporated to dryness affording 58.8 g (98.7%) crude 12 as a pale yellow liquid. Vacuum distillation over a 10 cm Vigreux column afforded 54.5 g (91.6%) Weinreb-amide 12 as a colorless oil, bp. 59-60° C./0.5 mbar. ¹H NMR (CDCl₃, 400 MHz) δ 3.10 (s, 1H), 3.25 (s, 3H), 3.70 (s, 3H), 4.29 (s, 2H).b5) N-Methoxy-2-methoxymethoxy-N-methyl-acetamide

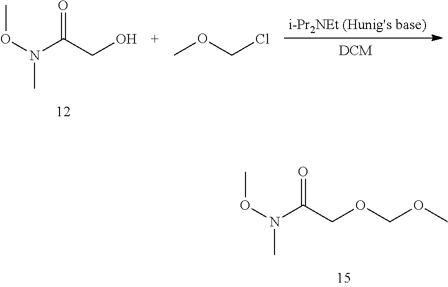

To a solution of Weinreb-amide 12 (59.6 g, 500 mmol) in dichloromethane (500 ml) was added while stirring N-ethyldiisopropylamine (96.9 g, 750 mmol) and the colorless solution was cooled to 0° C. chloromethyl methyl ether (50.3 g, 625 mmol) was added at 0° C. over 15 min and the reaction mixture was stirred at 0° C. for 1 h and at RT for three days. The pale yellow reaction mixture was washed with 1M HCl (500 ml) and 5% brine (500 ml) and the aqueous layers were extracted with dichloromethane (250 ml). The combined organic layers were dried (Na₂SO₄) and evaporated to dryness affording 78.9 g (96.7%) crude 15 as a yellow liquid. Vacuum distillation over a 10 cm Vigreux column afforded 74.4 g (91.2%) Weinreb-amide 15 as a colorless oil, bp. 80° C./0.5 mbar. ¹H NMR (CDCl₃, 400 MHz) δ 3.20 (s, 3H), 3.42 (s, 3H), 3.70 (s, 3H), 4.37 (s, 2H), 4.75 (s, 2H).

### b6) N-Methoxy-N-methyl-2-(tetrahydro-pyran-2-yloxy)-acetamide

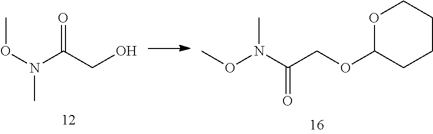

Alcohol 12 (26.6 g, 222 mmol, 1.00 equiv) was dissolved in dichloromethane (270 ml) Pyridinium para-toluene sulfonic acid (565 mg, 2.22 mmol, 0.01 equiv) was added followed by dihydropyran (28.3 ml, 309 mmol, 1.39 equiv) and the resulting mixture was stirred at ambient temperature for 19 hours. The mixture was transferred into a separatory funnel and washed with 5% aqueous sodium bicarbonate solution (270 ml). The aqueous layer was extracted with dichloromethane (100 ml) and the combined organic layers were dried over anhydrous Na2SO4, filtered, and concentrated under reduced pressure. The title compound 16 was obtained in quantitiative yield as clear, colorless oil.

¹H NMR (400 MHz, CDCl₃) δ 4.78-4.74 (m, 1H), 4.48-4.35 (m, 2H), 3.93-3.84 (m, 1H), 3.69 (s, 3H), 3.56-3.49 (m, 1H), 3.19 (s, 3H), 1.95-1.49 (m, 6H).

### c1) N-[5-(2-tert-Butoxy-acetyl)-pyrazin-2-yl]-2,2-dimethyl-propionamide

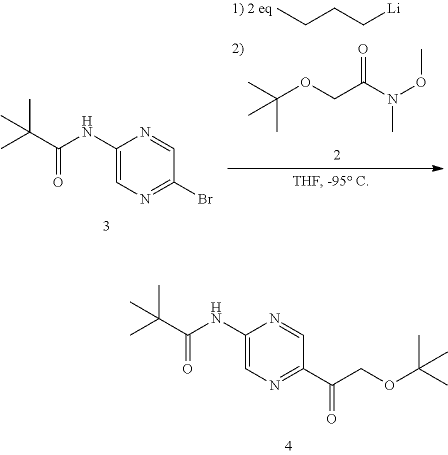

2.7 M Butyllithium in heptane (39 m≅27.6 g, 105.0 mmol) was added at −95 to −100° C. to a solution of N-(5-Bromo-pyrazin-2-yl)-2,2-dimethyl-propionamide 3 (12.9 g, 50 mmol) in 400 ml THF as follows: the first 19 ml (˜1.02 eq.) were added at −95° C. over 60 min and the remaining 19 ml (˜1.02 eq.) over 15 min. After stirring at −95° C. for 15 min the cooling bath was removed and Weinrebamide 2 (8.8 g, 50 mmol) was added over 5 min under vigorous stirring to the red solution whereby the reaction mixture warmed up to −60° C. and became a viscous suspension. Stirring at −50° C. was continued for 1 h and the reaction mixture was hydrolyzed with 2 M HCl (75 ml, ˜150 mmol). The yellow organic layer was washed with 10% brine (100 ml), dried (Na₂SO₄) and evaporated to afford 15.1 g (“103.0%”) beige crystalline residue which was dissolved in 2-butanol (60 ml) at ˜65° C. and crystallized by cooling to RT and at −20° C. 12.6 g (85.9%) ketone 4 as a white powder, mp. 138-140° C. ¹H NMR (CDCl₃, 400 MHz) δ 1.31 & 1.36 (s, each 9H), 4.96 (s, 2H), 8.14 (br s, 1H), 8.92 (d, J=1.6 Hz, 1H), 9.55 (d, J=1.3 Hz, 1H).

### c2) N-[5-(2-Methoxymethoxy-acetyl)-pyrazin-2-yl]-2,2-dimethyl-propionamide

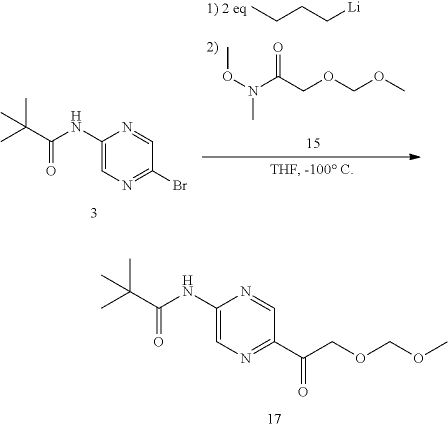

2.7 M Butyllithium in heptane (39 ml≅27.6 g, 105.0 mmol) was added at −95 to −100° C. to a solution of N-(5-Bromo-pyrazin-2-yl)-2,2-dimethyl-propionamide 3 (12.9 g, 50 mmol) in THF (400 ml) as follows: the first 19 ml (˜1.02 eq.) were added over 60 min and the remaining 19 ml (˜1.02 eq.) over 15 min. After stirring at −95° C. to −100° C. for 15 min, the cooling bath was removed and Weinrebamide 15 (8.2 g, 50 mmol) was added over 5 min under vigorous stirring to the red solution whereby the reaction mixture warmed up to −60° C. and became a viscous suspension. Stirring at −50° C. was continued for 1 h and the reaction mixture was hydrolyzed with 2 M HCl (75 ml, ˜150 mmol). The yellow organic layer was washed with 10% brine (100 ml), dried (Na₂SO₄) and evaporated to afford 15.1 g beige crystalline residue which was dissolved in 2-butanol (105 ml) at ˜75° C. and crystallized by cooling to RT and to −20° C. 11.6 g (82.4%) ketone 17 as a white powder, mp. 141-142° C.

¹H NMR (CDCl₃, 400 MHz) δ 1.36 (s, each 9H), 3.44 (s, 3H), 4.81 (s, 2H), 5.11 (s, 2H), 8.15 (br s, 1H), 8.93 (d, J=1.6 Hz, 1H), 9.55 (d, J=1.3 Hz, 1H)

### c3) 2,2-Dimethyl-N-{5-[2-(tetrahydro-pyran-2-yloxy)-acetyl]-pyrazin-2-yl}-propionamide

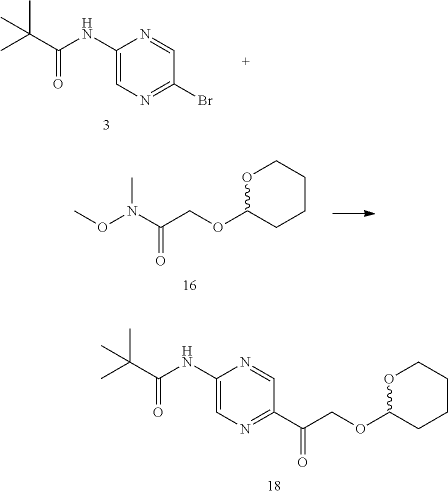

In a 750-ml 4-necked flask equipped with a Pt-100 thermometer, a mechanical stirrer, an addition funnel with inert gas supply, and a rubber septum, bromide 3 (10.3 g, 40.0 mmol) was dissolved in THF (320 ml). The solution was cooled to −78° C. and n-butyllihium (2.7 M in heptane, 31 ml, 2.1 equiv) was added via syringe pump over 30 minutes. 15 minutes after the end of the addition, Weinreb amide 16 (8.38 g, 40.0 mmol, 1.00) was added dropwise at −78° C. over 15 minutes. 30′ after the end of the addition, 1 M aqueous hydrochloric acid (200 ml) was added over 1 minute, allowing the reaction mixture to warm up to ca. 0° C. The layers were separated and the aqueous phase was extracted with ethyl acetate (200 ml). The combined organic phases were washed with 5% aqueous sodium bicarbonate solution (200 ml) and then with 10% aqueous sodium chloride solution (200 ml). The organic phase was dried over anhydrous sodium sulfate, filtered, and concentrated under reduced pressure to afford 12.6 g of the crude title compound as a yellowish solid.

The crude product was suspended in 2-butanol (50 ml) and stirred for 3 hours at 60° C. The mixture was allowed to cool to ambient temperature and was stirred at 20° C. for 65 hours and at 0° C. for 6 hours. The crystals were filtered off, washed with 2-butanol (10 ml), and dried under reduced pressure (10 mbar) at 50° C. for 17 hours. 6.95 g (54% yield) of the title compound 18 were isolated as white crystals.

¹H NMR (600 MHz, CDCl₃) δ 9.56 (d, 1H), 8.92 (d, 1H), 8.13 (br s, 1H), 5.21 (d, 1H), 5.10 (d, 1H), 4.82-4.78 (m, 1H), 3.95-3.88 (m, 1H), 3.58-3.50 (m, 1H), 2.00-1.50 (m, 6H), 1.36 (s, 9H). MS: m/e 322 (M+H)⁺, 238.

### c4) N-[5-(2-Hydroxy-acetyl)-pyrazin-2-yl]-2,2-dimethyl-propionamide

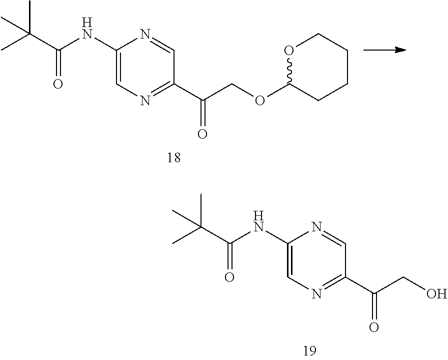

In a 250-ml 3-necked flask equipped with a mechanical stirrer, a Pt-100 thermometer, and an inert gas supply, THP ether 18 (19.1 g, 59.6 mmol) is suspended in 2-butanol (150 ml) and pyridinium para-toluenesulfonate (77 mg, 0.5 mol %) is added. The mixture is stirred at 70° C. for 3 hours and allowed to cool to ambient temperature. After stirring for 3 hours at ambient temperature and 18 hours at 0° C., the crystals were filtered off, washed with ice-cooled 2-butanol (23 ml), and dried under reduced pressure (10 mbar) at 50° C. for 21 hours. 13.3 g (94% yield) of the title compound are obtained as white crystals.

¹H NMR (600 MHz, CDCl₃) δ 9.60 (d, 1H), 8.95 (d, 1H), 8.12 (br s, 1H), 5.04 (d, 2H), 3.29 (t, 1H), 1.37 (s, 9H); MS: m/e 238 (M+H); m.p=169-172° C.

### Step b

### Enzymatic or Microbial Asymmetric Reduction by an Oxidoreductase

### Example 2

### a1) N-[5-((S)-2-tert-Butoxy-1-hydroxy-ethyl)-pyrazin-2-yl]-2,2-dimethyl-propion-amide

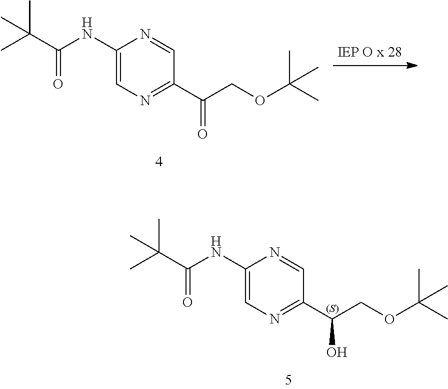

NAD-cofactor (20 mg) was dissolved in buffer (120 ml; 100 mM triethylamine buffer pH 9.0 containing 1 mM magnesium chloride and 10% glycerol) and the solution mixed with 2-propanol (80 ml). tert-Butoxy ketone (40 g) and oxidoreductase IEPox28 (23 ml cell suspension, 200 g cells/L; containing 50% glycerol; for preparation see note below) was added to the solution under vigorous stirring. The reaction mixture was stirred for 70 h at 30° C. After termination of the reaction, excess 2-propanol and formed acetone was removed by evaporation and the residual aqueous phase extracted with ethyl acetate (3×250 ml). The combined organic layers were dried (Na₂SO₄) and the solvent was evaporated affording 43.9 g (109.0%) crude alcohol 5 as a yellow solid which was used without purification in the next step (>99% ee (Column: Chiralpak IA, 5 μm, 250×4.6 mm (Daicel); flow: 1.0 ml/min; 40° C.; pressure: 43 bar; mobile phase: 90% TBME, 5% DEA (0.1%) in TBME, 5% EtOH; detection: 238 nm; injection: 1 μl; concentration: 2 mg/ml in EtOH; retention time: (S)-alcohol: 5.07 min, ketone: 6.49 min, (R)-alcohol: 6.98 min)).

A sample was purified by crystallization from methylcyclohexane for the measurement of the optical rotation and the NMR: [a]_{D}²⁰=39.3 (c 1.0; CHCl₃), ¹H NMR (CDCl₃, 400 MHz) δ 1.17 (s, 9H), 1.35 (S, 9H), 3.42 (d, J=5 Hz, 1H), 3.56 and 3.65 (ABX, J_{AB}=9 Hz, J_{AX}=7 Hz, J_{BX}=5 Hz, each 1H), 4.89 (m, 1H), 7.94 (br s, 1H), 8.43 (d, J=1.6 Hz, 1H), 9.48 (d, J=1.3 Hz, 1H).

Note: The E. coli strain RB791 containing oxidoreductase IEPox28 is deposited at Deutsche Sammlung von Mikroorganismen and Zellkulturen (DSMZ; DSM-No. 22053) and was cultivated in LB-medium (200 ml; 1% tryptone, 0.5% yeast extract, 1% sodium chloride) containing kanamycin (50 μg/ml) until an optical density of 0.5 was achieved (550 nm). The expression of recombinant protein was induced by addition of isopropyl thiogalactoside (0.1 mM). After 12-16 h induction at 25° C. and 220 rpm the cells were harvested (centrifugation) and frozen at −20° C. For the transformation the cells were applied as a suspension, e.g. frozen cells (35 g) resuspended in 100 mM triethylamine buffer pH 7 containing 2 mM magnesium chloride (15 ml) and subsequently mixed with glycerol (50 ml).

### a2) N-[5-((S)-2-tert-Butoxy-1-hydroxy-ethyl)-pyrazin-2-yl]-2,2-dimethyl-propion-amide (Oxidoreductase IEP Ox19 (DSM 22167)

In an Eppendorf tube a mixture of buffer (250 μl; 100 mM triethylamine pH 7 containing 10% glycerol and 1 mM magnesium chloride), 2-octanol (250 μl), substrate (25 mg), NADPH (0.02 mg) and oxidoreductase IEPox 19 (75 μl cell suspension, 10% re E. coli; cells deposited at DMSZ (DSM-No. 22167); preparation of cells in analogy to note above) was incubated for 48 h at 25° C. According to HPLC the substrate was converted to the desired (S)-alcohol in >90%. a3) N-[5-((S)-2-tetrahydropyranyloxy-1-hydroxy-ethyl)-pyrazin-2-yl]-2,2-dimethyl-propion-amide (Oxidoreductase IEP Ox28 (DSM 22053))

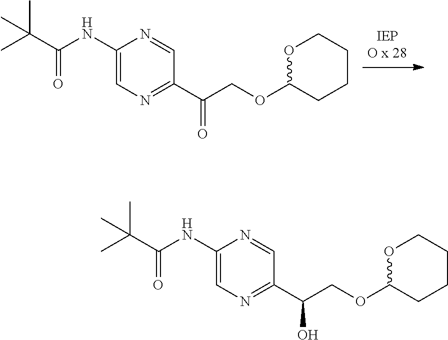

In an Eppendorf tube a mixture of buffer (300 μl; 100 mM triethylamine pH 8 containing 10% glycerol and 1 mM magnesium chloride), 2-propanol (200 μl), substrate (100 mg), NADH (0.05 mg) and oxidoreductase IEPox 28 (80 μl cell suspension, cells deposited at DMSZ (DSM-No. 22053), preparation of cells in analogy to note above) was incubated for 24 h at 50° C. According to HPLC the substrate was converted to the two desired (S)-alcohol diastereoisomers almost quantitatively (Column: Chiralcel OD-H 5 μm, 250×4 mm (Daicel); flow: 0.5 ml/min; pressure: 22 bar; mobile phase: 88% n-hexane, 12% 2-propanol; detection: 250 nm; injection: 10 μl; concentration: 0.5-1 mg/ml in n-hexane/2-propanol (90:10); retention time: Ketones: 24.2+25.8 min, (S)-alcohols: 15.9+16.7 min, (R)-alcohols: 19.2+20.2 min).

### a4) N-[5-((S)-2-methoxymethyloxy-1-hydroxy-ethyl)-pyrazin-2-yl]-2,2-dimethyl-propion-amide (Oxidoreductase IEP Ox28 (DSM 22053))

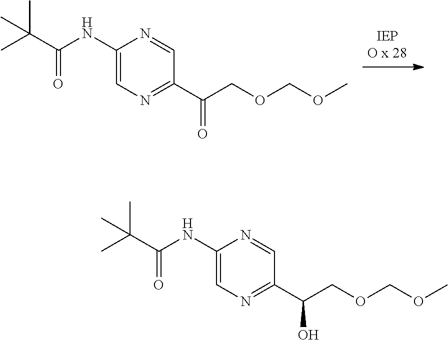

In an Eppendorf tube a mixture of buffer (250 μl; 100 mM triethylamine pH 8 containing 10% glycerol and 1 mM magnesium chloride), 2-propanol (250 μl), substrate (100 mg), NADH (0.04 mg) and oxidoreductase IEPox 28 (60 μl cell suspension, cells deposited at DMSZ (DSM-No. 22053), preparation of cells in analogy to note above) was incubated for 72 h at 25° C. According to HPLC the substrate was converted to the desired (S)-alcohol almost quantitatively (Column: Chiralcel OD-H 5 μm, 250×4 mm (Daicel); flow: 0.5 ml/min; pressure: 24 bar; mobile phase: 82% n-hexane, 18% 2-propanol; detection: 250 nm; injection: 10 μl; c concentration: 0.5 mg/ml in n-hexane/2-propanol (90:10); retention time: ketone: 22.9 min, (S)-alcohol: 13.0 min, (R)-alcohol: 15.4 min).

### a5) N-[5-((S)-1,2-dihydroxy-ethyl)-pyrazin-2-yl]-2,2-dimethyl-propion-amide (Codexis KRED-NADPH-101)

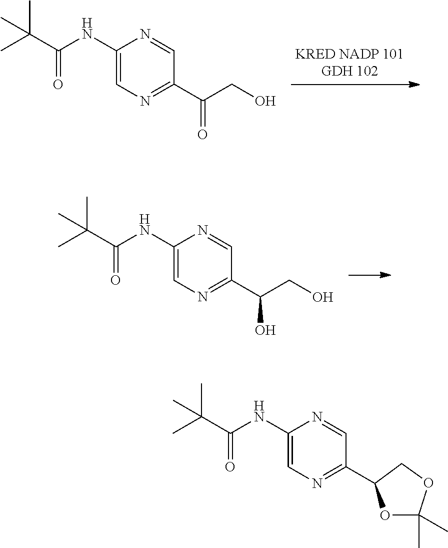

A mixture of buffer (70 ml 10 mM 2-(N-morpholino)ethane sulfonic acid pH 6.5 containing 10 g D-glucose monohydrate, 10 g polyethylene glycol 6000) and 10 g substrate was heated to 37° C. under stirring in a 250 ml flask. The pH was re-adjusted to pH 6.5, before the reaction was started by the addition of 40 mg NADP, 3.3 mg glucose dehydrogenase (GDH 102 from Biocatalytics [now Codexis]) and 20 mg ketoreductase (KRED-NADP-101 from Biocatalytics [now Codexis]). The pH was kept constant by addition of 1 M NaOH until complete conversion within 18 h. The product was extracted three times with 34 ml ethyl acetate after the addition of 30 g sodium chloride. The combined organic phases were dried over sodium sulfate and evaporated to a final volume of 80 ml. The enantiomeric excess of the diol was >99.9% (Column: Chiralcel AD-H 5 μm, 250×4.6 mm (Daicel); 40° C.; flow: 1 ml/min; pressure: 100 bar; mobile phase: 10% methanol, 90% ethanol; detection: 210 nm; injection: 5 μl; concentration: 2 mg/ml in ethanol; retention time: (R)-alcohol: 10.38 min, (S)-alcohol: 11.37 min).

Subsequently, the ketalization (step c) was immediately performed applying 29 ml 2,2-dimethoxy propane and 65 mg para-toluene sulfonic acid monohydrate at room temperature. After 4.5 h the reaction mixture was washed with 75 ml saturated sodium bicarbonate solution and 55 ml 100 mM phosphate buffer. The ethyl acetate was completely removed by evaporation. The crystals obtained were treated with heptane, which was directly removed by evaporation. The crystals obtained were dissolved in 149 ml TBME at 55° C. The solution was concentrated to ˜46 ml and allowed to cool to room temperature under stirring over night. The temperature was further decreased to approximately −9° C. for 2 h, before the product was isolated by filtration yielding 9.34 g white crystals. Analysis: purity 99.8%; ee>99.9% (Column: Chiralpak IA 5 μm, 250×4.6 mm (Daicel); flow: 1 ml/min; pressure: 70 bar; mobile phase: 50% acetonitrile, 50% ethanol; detection: 240 nm; injection: 10 μl; concentration: 1 mg/ml in ethanol; retention time: (R)-alcohol: 7.56 min, (S)-alcohol: 11.73 min); [α]_{d}=+76.5 (EtOH; c=1); ¹H NMR (DMSO, 400 MHz) δ 1.25 (s, 9H), 1.41 & 1.45 (s, each 3H), 3.95 (dd, J₁=14 Hz, J₂=11 Hz, 1H), 4.37 (dd, J₁=9 Hz, J₂=12 Hz, 1H), 5.18 (t, J=9 Hz, 1H), 8.47 (d, J=2 Hz, 1H), 9.48 (d, J=2 Hz, 1H), 10.30 (s, 1H); MS 280.1 M+H.

### Catalytic Asymmetric Hydrogenation Using a Metal Complex Catalyst

### Abbreviations:

tBu=t-butyl

### Ipr=Isopropyl

### MeO=Methoxy

### OTf=CF₃SO₃⁻

COD=1,5-cyclooctadiene
BARF=tetrakis[3,5-bis(trifluoromethyl)phenyl]borate
S/C=substrate-to catalyst molar ratio
Ferrocenyl phosphine ligands of the Josiphos family and ligands of the C3-Tunaphos family are commercially available from Solvias AG, CH-4002 Basel. All MeOBIPHEP type of ligands are either commercially available from Solvias AG, CH-4002 Basel or can be prepared according to the examples or methods as described in patent application documents EP 0 398 132, WO 92/16535, EP 0 104 375 or EP 0 580 331. Segphos derivatives are commercially available from Takasago Int. Corp., 4 Volvo Drive, Rockleigh, N.J. 07647-0932. Tangphos is commercially available from Chiral Quest, Princeton Corporate Plaza, Monmouth Jct., NJ08852, USA. CatASium T2 is commercially available from Evonik (Degussa). (R,R)-2,3-Bis(PMetBu)-quinox is commercially available from Johnson Matthey, 28 Cambridge Science Park, Milton Road, Cambridge, CB4 0FP UK.

The following list provides the chemical names for the acronyms of the chiral phosphine ligands used:

| Acronyms | Chemical Name |
|---|---|
| 3,5-tBu-4-MeO-MeOBIPHEP | (6,6′-Dimethoxybiphenyl-2,2′-diyl)bis[bis(3,5-tert-butyl-4- |
| | methoxy-phenyl)phosphine] |
| 3,5-tBu-C3-TUNAPHOS | 6,6′-O-[1,3-propylene]-oxybiphenyl-2,2′-diyl)bis(di(3,5-di-tert- |
| | Butylphenyl)phosphine |
| 3,5-Ipr,4-DMA-MeOBIPHEP | (6,6′-Dimethoxybiphenyl-2,2′-diyl)bis[bis(3,5-iso-propyl-4- |
| | dimethylamino)-phenyl)phosphine] |
| 3,4,5-MeO-TriMeOBIPHEP | (6,6′-Dimethoxybiphenyl-2,2′-diyl)bis[bis(3,4,5-tri-methoxy- |
| | phenyl)phosphine] |
| 3,5-Ipr,4-MeOBIPHEP | (6,6′-Dimethoxybiphenyl-2,2′-diyl)bis[bis(3,5-iso-propyl-4- |
| | methoxy)-phenyl)phosphine] |
| 3,5-MOR-MeOBIPHEP | (6,6′-Dimethoxybiphenyl-2,2′-diyl)bis[bis(3,5-morpholino)- |
| | phenylphosphine] |
| DTBM-SEGPHOS | 5,5′-Bis[di(3,5-di-tert-butyl-4-methoxyphenyl)phosphino]-4,4′- |
| | bi-1,3-benzodioxole |
| 2,3-Bis(PMetBu)-quinox | 2,3-Bis(tert-butylmethylphosphino)quinoxaline |
| 3,5-tBu-4-MeO-MeOBIPHEP | (6,6′-Dimethoxybiphenyl-2,2′-diyl)bis[bis(3,5-tert-butyl- |
| | phenyl)phosphine] |
| catASium T2 | 3-Di-(3,5-diemthylphenyl)phosphino-(4-diphenylphosphino- |
| | 2,5-dimethylthienyl-3)-1,7,7-trimethylbicyclo[2.2.1]heptane-2 |
| Tangphos | 1,1′-Di-tert-butyl-[2,2′]-diphospholane |
| tBu-Josiphos | 1-[Bis(1,1-dimethylethyl)phosphino]ethyl]-2-(diphenyl |
| | phosphino)ferrocene |

### Example 3

a) In a glove box (O₂ content≦2 ppm) a 35 ml autoclave with glass insert and magnetic stirring bar was charged with 0.3 g (1.023 mmol) of N-[5-(2-tert-Butoxy-acetyl)-pyrazin-2-yl]-2,2-dimethyl-propionamide, 0.42 mg (0.010 mmol, S/C 1000) of [Rh(COD)₂]BF₄, 1.30 mg (0.0011 mmol) (S)-3,5-tBu,4-MeO-MeOBIPHEP (catalyst type XXe/Rh-1) and 6 ml of ethanol. The asymmetric hydrogenation was run for 17 h at 80° C. under 50 bar of hydrogen. After cooling to room temperature the pressure was released from the autoclave, the solvent was removed under vacuum to give N-[5-(S)-2-tert-Butoxy-1-hydroxy-ethyl)-pyrazin-2-yl]-2,2-dimethyl-propionamide in quantitative yield and with 94.2% ee as light yellow oil, which solidified upon standing.

### b1)-b11): [Rh(COD)₂]BF₄ als pre-cursor^{a}

| | Ligand - Catalyst | | | | | | |
|---|---|---|---|---|---|---|---|
| Example | Type | S/C | Solvent | T[° C.] | p [bar] | Purity | ee |
| b1^{b} | 3,5-tBu,4-MeO- | 1500 | EtOH | 80 | 50 | >98 | 93.2/S |
| | MeOBIPHEP - | | | | | | |
| | XXe/Rh-1 | | | | | | |
| b2 | 3,5-tBu,4-MeO- | 50 | EtOH | 50 | 50 | >99.9 | 96.2/S |
| | MeOBIPHEP - | | | | | | |
| | XXe/Rh-1 | | | | | | |
| b3^{c} | 3,5-tBu,4-MeO- | 100 | CH₂Cl₂ | 50 | 50 | >99.9 | 95.2/S |
| | MeOBIPHEP - | | | | | | |
| | XXe/Rh-1 | | | | | | |
| b4 | (S)-3,5-tBu-C3- | 50 | EtOH | 50 | 50 | >99.9 | 94.4/S |
| | TUNAPHOS - | | | | | | |
| | XXe/Rh-1 | | | | | | |
| b5 | (R,S)-tBu-Josiphos - | 50 | CH₂Cl₂ | 50 | 50 | 98 | 67.4/S |
| | XXe/Rh-2 | | | | | | |
| b6 | (S)-3,5-Ipr,4-DMA- | 50 | CH₂Cl₂ | 50 | 50 | >99.9 | 93.6/S |
| | MeOBIPHEP - | | | | | | |
| | XXe/Rh-1 | | | | | | |
| b7 | (S)-3,4,5-MeO- | 50 | CH₂Cl₂ | 50 | 50 | >99.9 | 90.6/S |
| | TriMeOBIPHEP - | | | | | | |
| | XXe/Rh-1 | | | | | | |
| b8 | (S)-3,5-MOR- | 50 | CH₂Cl₂ | 50 | 50 | >99.9 | 87.4/S |
| | MeOBIPHEP - | | | | | | |
| | XXe/Rh-1 | | | | | | |
| b9 | (S)-3,5-Ipr,4- | 50 | CH₂Cl₂ | 50 | 50 | >99.9 | 92.4/S |
| | MeOBIPHEP - | | | | | | |
| | XXe/Rh-1 | | | | | | |
| b10 | (S)-DTBM- | 50 | CH₂Cl₂ | 50 | 50 | >99.9 | 90.8/S |
| | SEGPHOS - | | | | | | |
| | XXe/Rh-1 | | | | | | |
| b11 | (R,R)-2,3- | 50 | CH₂Cl₂ | 50 | 50 | >99.9 | 80.4/R |
| | Bis(PMetBu)- | | | | | | |
| | quinox - XXe/Rh-3 | | | | | | |

^{a}Conditions: 6 ml autoclave, 50 mg (0.17 mmol) scale, solvent (1 ml, 3.6-5.9 w %), 17 h.
^{b}35 ml autoclave, 0.3 g (1.023 mmol) scale, EtOH (6 ml, 5.9 w %), 17 h.
^{c}35 ml autoclave, 0.5 g (1.704 mmol) scale, CH₂Cl₂ (6 ml, 5.9 w %), 17 h.

### c1)-c5): Isolated Complexes^{a}

| Example | Rh complex - Catalyst Type | S/C | T [° C.] | p [bar] | Purity | ee |
|---|---|---|---|---|---|---|
| c1 | [Rh(COD)((S)-3,5-tBu,4-MeO- | 500 | 80 | 20 | >99 | 93.0/S |
| | MeOBIPHEP)]OTf - XXe/Rh-5 | | | | | |
| c2 | [Rh(COD)((S)-3,5-tBu,4-MeO- | 500 | 80 | 40 | >99 | 93.2/S |
| | MeOBIPHEP)] OTf - XXe/Rh-5 | | | | | |
| c3^{b} | [Rh(COD)((S)-3,5-tBu- | 50 | 50 | 50 | 99 | 91.0/S |
| | MeOBIPHEP)]BARF - XXe/Rh-4 | | | | | |
| c4^{c} | [Rh(COD)((R)-catASium)]OTf | 50 | 50 | 50 | >99.9 | 64.4/R |
| | XXe/Rh-6 | | | | | |
| c5^{c} | [Rh(COD)((S,S,R,R)- | 50 | 50 | 50 | >99.9 | 87.0/R |
| | Tangphos)]BF₄ - XXe/Rh-7 | | | | | |

^{a}Conditions: 35 ml autoclave, 50 mg (0.17 mmol) scale, EtOH (2 ml, 3.1 w %), 17 h.
^{b}CH₂Cl₂ (1 ml, 3.6 w %) as solvent,.
^{c}Conditions: 6 ml autoclave, 50 mg scale, CH₂Cl₂ (1 ml, 3.6 w %), 17 h.

### d) N-[5-((S)-2-tert-Butoxy-1-hydroxy-ethyl)-pyrazin-2-yl]-2,2-dimethyl-propionamide ([Ir(COD)((S)-3,5-tBu-MeOBIPHEP)]BARF)

In a glove box (O₂ content≦2 ppm) a 35 ml autoclave with glass insert and magnetic stirring bar was charged with 0.05 g (0.170 mmol) of N-[5-(2-tert-Butoxy-acetyl)-pyrazin-2-yl]-2,2-dimethyl-propionamide, 7.48 mg (0.0034 mmol, S/C 50) of [Ir(COD)((S)-3,5-tBu-MeOBIPHEP)]BARF (catalyst type XXe/Ir-1) and 1 ml of dichloromethane. The asymmetric hydrogenation was run for 17 h at 50° C. under 50 bar of hydrogen. After cooling to room temperature the pressure was released from the autoclave, the solvent was removed under vacuum to give N-[5-((S)-2-tert-Butoxy-1-hydroxy-ethyl)-pyrazin-2-yl]-2,2-dimethyl-propionamide in quantitative yield and with 84.6% ee as light yellow oil.

### e) N-[5-((S)-2-methoxymethyloxy-1-hydroxy-ethyl)-pyrazin-2-yl]-2,2-dimethyl-propion-amide ([Rh(COD)((S)-3,5-tBu,4-MeO-MeOBIPHEP)]BF₄)

In a glove box (O₂ content≦2 ppm) a 35 ml autoclave was charged with 0.3 g (1.066 mmol) of N-[5-(2-Methoxymethoxy-acetyl)-pyrazin-2-yl]-2,2-dimethyl-propionamide, 0.43 mg (0.011 mmol, S/C 1000) of [Rh(COD)₂]BF₄, 1.35 mg (0.0012 mmol) (S)-3,5-tBu,4-MeO-MeOBIPHEP (catalyst type XXe/Rh-1) and 6 ml of trifluoroethanol. The asymmetric hydrogenation was run for 18 h at 80° C. under 50 bar of hydrogen. After cooling to room temperature the pressure was released from the autoclave, the solvent was removed under vacuum to give N-[5-((S)-1-Hydroxy-2-methoxymethoxy-ethyl)-pyrazin-2-yl]-2,2-dimethyl-propionamide in 94% yield and with 93.2% ee as light yellow oil. ¹H-NMR (CDCl₃, 400 MHz): δ 9.49 (d, 1H, J=1.5 Hz), 8.43 (d, 1H, J=1.5 Hz), 7.98 (br s, 1H, NH), 4.98 (m, 1H), 4.67 (s, 2H), 3.93 (dd, 1H, J=4.0 Hz and 10.5 Hz), 3.8 (m, 2H), 1.35 (s, 9H).

### Step c

### Example 4

### N-[5-((S)-2,2-Dimethyl-[1,3]dioxolan-4-yl)-pyrazin-2-yl]-2,2-dimethyl-propionamide

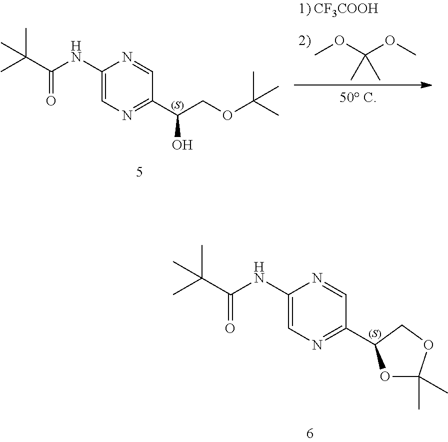

N-[5-((S)-2-tert-Butoxy-1-hydroxy-ethyl)-pyrazin-2-yl]-2,2-dimethyl-propion-amide 5 (6.44 g, theor. 20 mmol) were dissolved in trifluoroacetic acid (15.3 ml, 200 mmol) and the red solution was stirred at 50° C. for 2 h. After cooling to 5° C., 2,2-dimethoxypropane (49.0 ml, 400 mmol) were added and stirring at 50° C. was continued for 18 h. The red reaction mixture was cooled to RT and poured under stirring to a cold (˜−5° C.) mixture of dichloromethane (40 ml) and 3 M NaOH (67 ml, ˜200 mmol). The organic layer was washed with 10% brine (40 ml) and both aqueous layers were extracted with dichloromethane (40 ml). The combined organic layers were dried (Na₂SO₄) and evaporated to dryness (35-50° C./≧10 mbar) affording 6.28 g (“112%”) brown, crystalline residue which was dissolved in refluxing isopropyl ether (80 ml) to yield after cooling and stirring at −20° C. for 16 h 4.78 g (85.6%) acetonide 6 as a off-white powder, mp. 142-143° C., [α]_{d}=+86.2 (CHCl₃; c=1). ¹H NMR (CDCl₃, 400 MHz) δ 1.35 (s, 9H), 1.50 & 1.54 (s, each 3H), 4.01 (dd, J₁=7 Hz, J₂=9 Hz, 1H), 4.45 (dd, J₁=7 Hz, J₂=9 Hz, 1H), 5.22 (t, J=7 Hz, 1H), 7.94 (br s, 1H), 8.43 (d, J=1.6 Hz, 1H), 9.48 (d, J=1.3 Hz, 1H).

### Step d

### Example 5

### 5-((S)-2,2-Dimethyl-[1,3]dioxolan-4-yl)-pyrazin-2-ylamine

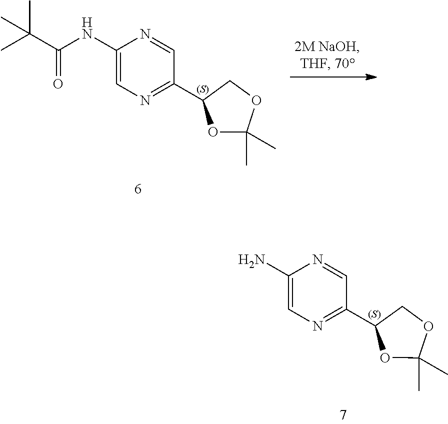

To a suspension of N-[5-((S)-2,2-Dimethyl-[1,3]dioxolan-4-yl)-pyrazin-2-yl]-2,2-dimethyl-propionamide 6 (27.9 g, 100 mmol) in THF (50 ml) was added 2M NaOH (75 ml, 150 mmol) and the biphasic reaction mixture was vigorously stirred under reflux for 18 h. After cooling to RT the reaction mixture was extracted with dichloromethane (200 & 2×100 ml). All three organic layers were washed sequentially with 5% brine (100 ml), combined and dried (Na₂SO₄). Evaporation of the solvent (35-50° C./≧10 mbar) afforded 19.40 g (99.4%) crude aminoketal 7 as a yellow viscous oil which was used without purification in the next step. ¹H NMR (CDCl₃, 400 MHz) δ 1.48 & 1.53 (s, each 3H), 4.00 (dd, J₁=6 Hz, J₂=8 Hz, 1H), 4.35 (dd, J₁=6 Hz, J₂=8 Hz, 1H), 4.62 (br s, 2H), 5.10 (t, J=6 Hz, 1H), 7.94 (d, J=1.3 Hz, 1H), 8.15 (d, 1.1 Hz, 1H).

### Step e

### Example 6

### a) (R)-2-(3-Chloro-4-methanesulfonyl-phenyl)-3-cyclopentyl-propionyl chloride

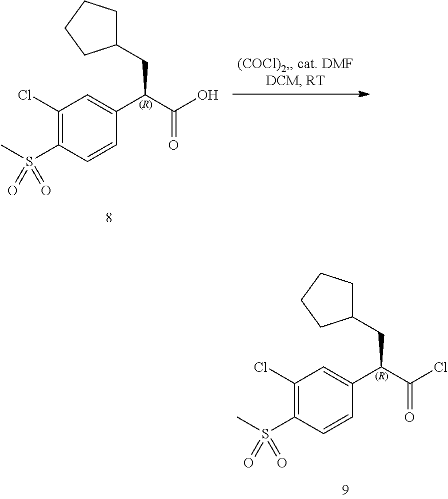

To the sulfone acid 8 (33.08 g, 100 mmol, er 99.3:0.7) dissolved in dichloromethane (250 ml) were added at RT DMF (0.15 ml, 2 mmol) and then a solution of oxalyl chloride (9.45 ml, 110 mmol) in dichloromethane (25 ml) over 10 min. After the colorless solution was stirred at RT for 3 h (a gas evolution was observed: CO & CO₂), the main part of the solvent (DCM) was removed at the rotary evaporator keeping the water bath at RT (20-25° C./≧10 mbar). The gummy residue was dissolved in dichloromethane (ca. 150 ml) and evaporated at RT as described above affording 38.1 g acid chloride 9 as a colorless gum which was immediately used in the next step or stored under Ar at ≦0° C. ¹H NMR (CDCl₃, 400 MHz) δ 1.13 (m, 2H), 1.40-1.85 (m, 7H), 1.90 and 2.22 (m, each 1H), 3.29 (s, 3H), 4.08 (t, 1H), 7.41 (dd, J₁=8 Hz, J₂=1.7 Hz, 1H), 7.50 (d, J=1.7 Hz, 1H), 8.16 (d, J=8 Hz, 1H).

### b) (R)-2-(3-Chloro-4-methanesulfonyl-phenyl)-3-cyclopentyl-N-[5-((S)-2,2-dimethyl-[1,3]dioxolan-4-yl)-pyrazin-2-yl]-propionamide

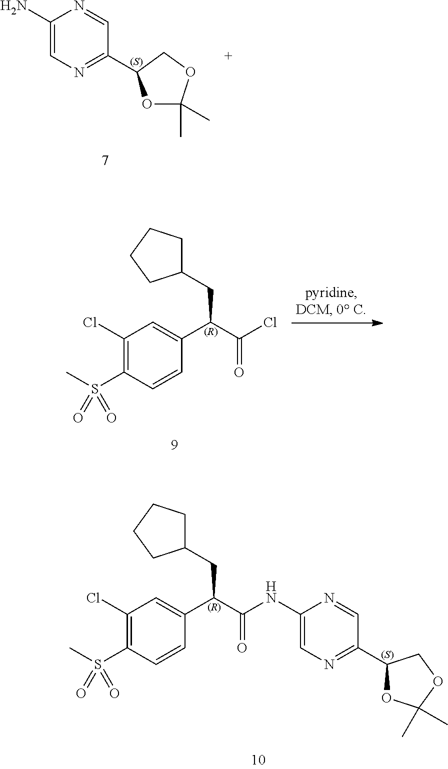

To the aminoketal 7 (19.4 g, theor. 100 mmol) dissolved in dichloromethane (200 ml) was added pyridine (9.7 ml, 120 mmol) and the reaction mixture was cooled to 0° C. A solution of the acid chloride 9 (38.1 g, theor. 100 mmol) in dichloromethane (100 ml) was added at 0° C. over ˜15 min and the colorless reaction mixture was stirred for additional 1.5 h at 0° C. and for 0.5 h at RT. The reaction mixture was cooled to 0° C. and hydrolyzed under vigorous stirring with 1M HCl (200 ml). The organic layer was separated and washed with 5% NaHCO₃ (200 ml), dried (Na₂SO₄) and evaporated carefully (35-50° C./≧10 mbar) affording 51.3 g (“101.0%”) crude amide 10 as a voluminous, white foam which was used without purification in the next step. ESI-MS (m/z): 508 (M+H⁺, 100).

### Step f

### Example 7

### a) (R)-2-(3-Chloro-4-methanesulfonyl-phenyl)-3-cyclopentyl-N-[5-((S)-1,2-dihydroxy-ethyl)-pyrazin-2-yl]-propionamide

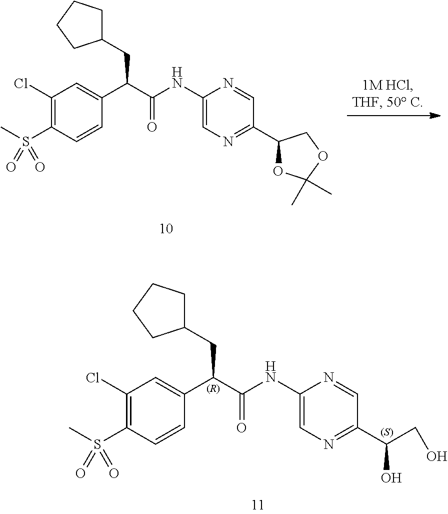

To a solution of the amide 10 (theor. 51.3 g, 100 mmol) in THF (200 ml) was added 1M HCl (250 ml) and the biphasic reaction mixture was stirred at 50° C. for 2.5 h. After cooling to RT the reaction mixture was extracted with dichloromethane (400 ml) and the organic layer was washed 3 times with 5% NaHCO₃ (3×300 ml) and once with 10% brine (300 ml). The organic layer was dried (Na₂SO₄) and evaporated carefully (35-50° C./≧10 mbar) affording 47.7 g (“101.9%”) crude 11 as a voluminous, white foam.

Crystallization: The above foam (47.7 g crude diol 11) was dissolved in isopropyl acetate (140 ml) at ˜60° C. and heptane (18.7 ml) was added drop wise under magnetical stirring over ˜5 min. After cooling to RT the clear solution was seeded with a suspension of 0.9 g pulverized, crystalline 11 in i-PrOAc/heptane=7.5:1 (ca. 2 ml) and stirring at RT was continued for 100 h. After additional stirring at −20° C. over night the white suspension was filtered, the filter cake washed with −20° C. cold i-PrOAc/heptane=7.5:1 (ca. 20 ml) and dried (50° C./10 mbar/4 h) affording 40.3 g (86%) 11 as a white crystalline powder, mp. 145-146° C. ESI-MS (m/z): 468 (M+H⁺, 100).

1. A process for the preparation of a compound of general formula I, 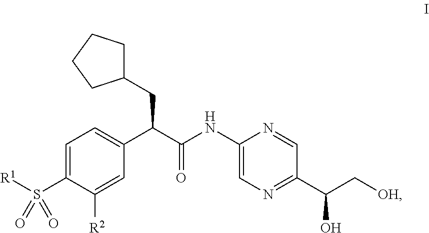
wherein R¹ is C₁₋₆-alkyl and R² is hydrogen or halogen, comprising the steps of
a) reacting a pyrazinamide of formula II, 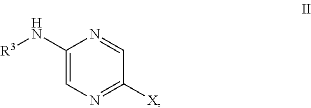
wherein R³ is an amino protecting group and X is halogen, with an amide compound of formula III, 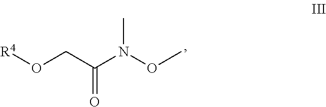
wherein R⁴ is a hydroxy protecting group, to form a ketone of formula IVa, 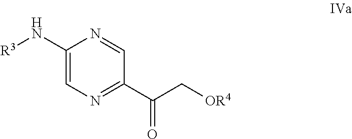
wherein R³ and R⁴ are as above;
b) optionally removing the R⁴ group to produce a ketone of formula IVb, 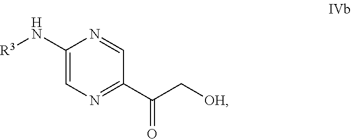
wherein R³ is as above, and asymmetrically reducing the compound of formula IVa or the compound of formula IVb to form the (S)-alcohol of formula V, 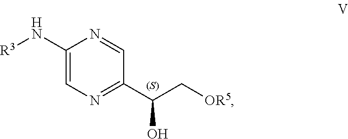
wherein R³ is as above and R⁵ is H or R⁴;
c) forming the acetonide of formula VI, 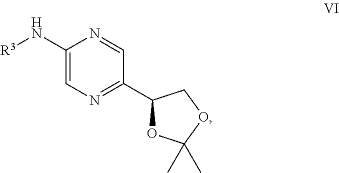
wherein R³ is as above;
d) removing R³ to form the amine of formula VII, 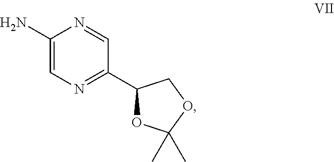
e) coupling said compound of formula VII with a (R)-2-phenyl propionic acid derivative of formula VIII, 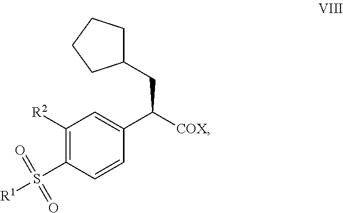
wherein R¹, R² and X are as above, to form the amide of formula IX, 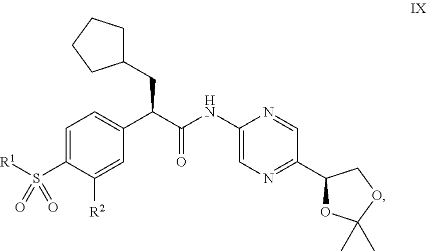
wherein R¹ and R² are as above; and
f) performing acidic acetonide hydrolysis to form the compound of formula I.

2. The process of claim 1, wherein R³ is pivaloyl.

3. The process of claim 1, wherein R⁴ is selected from the group consisting of: C₁₋₆-alkyl, C₁₋₆-alkylcarbonyl, C₁₋₆-alkoxy-C₁₋₆-alkyl and a saturated 5- or 6-membered heterocyclyl.

4. The process of claim 1, wherein the reaction of step a) is performed in the presence of a lithiating agent.

5. The process of claim 4, wherein said reaction is performed in an organic solvent at a reaction temperature of from −100° C. to −60° C.

6. The process of claim 1, wherein the asymmetric reduction is an enzymatic or microbial asymmetric reduction catalyzed by an oxidoreductase.

7. The process of claim 6, wherein the oxidoreductase is an NADH or NADPH dependent oxidoreductase.

8. The process of claim 6 wherein the oxidoreductase is selected from the group consisting of IEPox19, (DSM 22167), IEPox28 (DSM 22053), IEPox63 (DSM 22052), KRED 101 (Codexis) and oxidoreductase enzymes from Candida magnolia.

9. The process of claim 6 wherein said reduction is catalyzed in the presence of a co-factor and wherein said co-factor is regenerated by a secondary alcohol as co-substrate or glucose and glucose dehydrogenase.

10. The process of claim 1, wherein the asymmetric reduction is a catalytic reduction with a metal complex catalyst.

11. The process of claim 10, wherein the metal complex catalyst is selected from the group consisting of:
Ru(Z)₂D;
[Ru(Z)₂₋ₚ(D)(L)ₘ](Y)ₚ;
[Ru(D)(L)₂](Y)₂;
[M(D)LX]; and
[M(D)L]⁺Y⁻;
wherein
Z is selected from the group consisting of: hydrogen, halogen, η⁵-2,4-pentadienyl, η⁵-2,4-dimethyl-pentadienyl and the group A-COO⁻, wherein
A is selected from the group consisting of: C₁₋₆-alkyl, aryl, halogenated C₁₋₆-alkyl and halogenated aryl;
Y is a non-coordinating anion;
D is a chiral phosphine ligand;
L is a neutral ligand;
M is Iridium or Rhodium
X sis a halogen atom;
m is an integer from 1 to 3; and
p is 1 or 2.

12. The process of claim 11, wherein the metal complex catalyst is a compound of the formula
[M(D)L]⁺Y⁻,
wherein M, D, L and Y are as outlined above.

13. The process of claim 1, wherein the acetonide formation in step c) is performed by acidic treatment of the (S)-alcohol of formula V with trifluoroacetic acid and subsequent treatment with 2,2-dimethoxypropane.

14. The process of claim 1, wherein the removal of R³ in step d) is performed with a base.

15. The process of claim 14, wherein the base is selected from alcoholic solutions of an alkali carbonate or of an alkali hydroxide.

16. The process of claim 1, wherein the coupling in step e) is performed in an organic solvent in the presence of a tertiary amine.

17. The process of claim 1, wherein the acidic acetonide hydrolysis in step f) is performed with an aqueous mineral acid.

18. A compound of formula III, 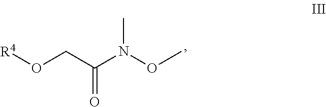
wherein R⁴ is t-butyl.

19. A compound of formula IVa, 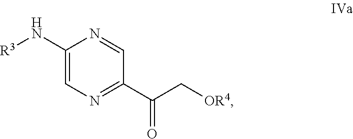
wherein R³ is an amino protecting group and R⁴ is a hydroxy protecting group.

20. The compound of claim 19, wherein R³ is pivaloyl and R⁴ is t-butyl.

21. The compound of formula V, 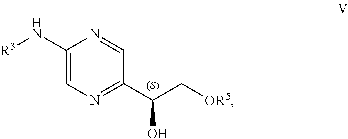
wherein R³ is an amino protecting group and R⁵ is H or is a hydroxy protecting group.

22. The compound of claim 21, wherein R³ is pivaloyl and R⁵ is H or t-butyl.
